(19) United States
(12) Patent Application Publication
(10) Pub. No.: US 2011/0184266 A1
(43) Pub. Date: Jul. 28, 2011

(54) Blood glucose monitoring system

(76) Inventor: Paul D. Levin, Santa Cruz, CA (US)

(21) Appl. No.: 12/657,639

(22) Filed: Jan. 25, 2010

(51) Int. Cl. A61B 5/145; A61M 5/14

(52) U.S. Cl. 600/365; 604/66

(57) ABSTRACT

A method and apparatus for blood glucose monitoring is provided which allows blood sampling and insulin infusion through the same catheter lumen of a multi-lumen, central venous catheter. It is designed to be used with a nearby continuous glucose sensor. The catheter lumen with the most proximal aperture leads to a connecting tube which is split into two parts, with both parts having a low internal volume. The described catheter allows for rapid switching between insulin delivery and blood sampling and minimizes the amount of purge fluid needed to clear the line.

### BACKGROUND AND SUMMARY OF THE INVENTION

A landmark study by Van den Berghe and colleagues, published in the Nov. 8, 2001 issue of the New England Journal of Medicine, showed improved outcomes in critically ill patients when blood glucose levels were kept in the normal range. Due to the release of stress hormones in very sick patients, or following major surgery or trauma, there is a natural tendency toward elevated blood sugars. Preventing hyperglycemia, without inadvertently causing hypoglycemia, is now the common goal in all hospital ICUs in the developed world.

An IV insulin infusion, most often given through one lumen of a central venous catheter, is universally used to suppress elevated blood sugar when it occurs, and the rate of infusion is judged from blood sugar measurements. Finger stick blood samples on an hourly basis and the use of a handheld glucometer is presently the most common method of tracking patient blood sugar levels. A system that could test blood sugar automatically without finger sticks would be a major improvement in the care of the critically ill. Besides avoiding the pain of finger sticks, it would give a more accurate picture of blood sugar levels by testing at least once every 10 minutes. Hourly tests are simply too infrequent to discern sudden rises or falls of blood glucose, which should be quickly adjusted for by changing the rate of the insulin infusion. Additionally, an automated system would save at least 40 minutes of a caregiver's time in a typical 8-hour nursing shift. Finally, there could be no contamination of personnel or equipment if finger stick samples were avoided.

FIG. 1 of this application shows a patient in a typical ICU environment with a nearby bedside, prior art glucose monitor (U.S. Pat. No. 7,162,290). A catheter 45 is inserted into a small blood vessel in the back of the patient's hand. Two bags 71,72 of solution, for high and low calibration of the sensor, are illustrated adjacent the monitor 70, along with a small glucose sensor 116 which is attached to the patient's forearm. This location for a catheter and a sensor is feasible only for those patients having reasonably large veins. Small peripheral veins, such as those in the back of the hand are often difficult to cannulate.

In contrast to the prior art system of FIG. 1, the present invention avoids the use of small, peripheral veins and/or finger sticks for obtaining blood samples. The present invention, for the first time, utilizes a single lumen of a central venous, multi-lumen catheter for obtaining blood samples for blood glucose monitoring and for infusing insulin solution. The preferred embodiment of the invention is shown in FIG. 12, described in detail below.

It would be much preferred by the medical staff of a typical hospital that a single lumen of the catheter be used for both the withdrawal of blood samples for testing and for the infusion of insulin to control blood sugar, leaving the other lumens available for other IV fluids. The present invention fulfills this preference of ICU caregivers.

Regardless of sensor location, in any system of blood withdrawal and return, a column of fluid in the sampling line is in continuity with the blood inside the venous catheter. Reversal of the peristaltic pumps (items 41 or 42 of FIG. 1) causes the fluid to pull blood into the sampling chamber of the glucose sensor (Item 116 of FIG. 1), but since the fluid in the line mixes with the blood being withdrawn, the fluid column must pull blood well past the sensor, approximately 20 cm, so that a pure blood sample is in the sensing area. A photodetector/LED pair inside the sensor determines the purity of the sample, as taught in U.S. Pat. No. 7,162,290. Because of blood/fluid intermixing, more fluid must be used to purge the system than was withdrawn. Therefore, with each test, extra fluid is added to the patient's circulation. In many ICU patients the circulatory system is already overloaded and additional fluid can worsen already present congestive heart failure. It is therefore most important to keep any added fluid to a minimum.

An inherent problem in attempting to utilize a single lumen for blood sample withdrawal and insulin infusion is that serious complications for the patient may occur with an interruption of insulin infusion for an extended period of time. It is absolutely critical to limit the time period or “cycle time” of the blood withdrawal, testing and return of the sample to the patient. The present invention achieves these goals as discussed below.

In most present day ICUs, one lumen of a triple lumen catheter is used for the patient's insulin infusion, leaving two lumens for other purposes. Ideally, an insulin infusion pump infuses fluid at a steady rate without peaks or valleys in the delivery of fluid to the patient. A typical infusion rate of a fluid with insulin is 3 mL/hr, but the rate can vary between 1 and 6 mL/hr depending on patient blood sugar levels. If a standard central venous catheter were to be used for both the insulin infusion and the withdrawal of blood samples, a time delay in insulin delivery would follow the return of each blood sample. This is because of the relatively large fluid capacity of typical catheters and specifically in their connecting tubes. It can be shown that if a standard catheter were to be used for both sampling and the infusion of insulin, at a rate of 3 mL/hr. between 8 and 10 minutes would elapse before insulin would appear at the aperture of the catheter after return of a test sample. When a new sample is taken during the cycle and then returned to the patient, all the insulin which has accumulated in the connecting tube is delivered to the patient as a bolus, along with the returned blood sample. A drop in blood sugar is likely from a sudden infusion of a large amount of insulin, which is undesirable. By contrast, the short period (2.5 minutes or less) of non-insulin infusion, when using the device of the present invention (FIG. 9), is not likely to cause this complication.

The present invention changes to some extent the design of the present day triple lumen catheters. It should be noted, however, that these changes are only to the connecting tubes (the “tails”) of the catheter and not to any feature inside of or pertaining to the inserted part of the catheter. A patent granted to Martin (U.S. Pat. No. 6,206,849) and a recent application by Markower (U.S. 2007/0208252) claim various new catheter features. These features involve only that part of the catheter that is inserted under the skin of the patient. Nowhere do either Markower or Martin show in the figures or describe in their patent texts any changes in the connecting tubing leading up to the inserted portions of the catheter. The Markower and Martin patents are concerned only with the design of those parts of the catheter under the patient's skin. For example, in column 5, lines 48-50 of the Martin patent, it is stated that “this lumen (best seen in FIG. 4) is an extension of the IV tubing and is proportioned to receive a 0.038 inch diameter Seldinger wire.” The lumen referred to is clearly the one inside the catheter proper and is not the lumen of a connecting tube. By contrast, the present invention changes features that are only outwardly of or external to the body or hub of the catheter.

A primary object of the invention is to provide a blood glucose monitoring system wherein only one lumen of a central venous multi-lumen catheter is used for both the blood sampling and for the infusion of insulin.

A further object of the invention is to alternate easily between blood sampling and insulin delivery.

A further object is to cause a minimal time delay in the delivery of insulin into the patient's central vein in order to withdraw, test and return a blood sample.

A further object is to minimize the infusion of additional purge fluid to clear the system after blood sampling.

A further object is to introduce modifications to a standard catheter without increasing the cost of manufacture.

### BRIEF DESCRIPTION OF THE DRAWINGS

FIG. 1 shows a prior art bedside system for long-term glucose monitoring with the sensor attached to the patient's forearm and an intravascular catheter in a dorsal hand vein.

FIG. 2 is a view of a standard triple lumen catheter as presently used in the subclavian vein.

FIG. 3 shows a standard triple lumen catheter attached to a test system for measuring purge volume.

FIG. 4 shows a modified triple lumen catheter as used in the test system. The connecting tubing leading to the proximal aperture of the catheter has an ID of 0.75 mm rather than the normal 1.5 mm but has the standard length of 13 cm.

FIG. 5 shows a modified catheter in which the connecting tubing leading to the proximal aperture has an ID of 1.5 mm but a length of only 3 cm.

FIG. 6 is a graph showing the test results from increasing the ID of a 13-cm connecting tubing in steps from 0.75 mm to 1.50 mm

FIG. 7 is a graph showing the test results of increasing the length of normal 1.50 mm ID tubing in steps from 3 cm to 13 cm.

FIG. 8 shows a test set-up for a simple length of Tygon tubing to determine the theoretical minimum purge volume.

FIG. 9 shows a triple lumen catheter with two changes in the connecting tubing to reduce purge volume to a minimum.

FIG. 10 is a chart showing the results from testing for purge volume in standard and modified central venous catheters.

FIG. 11 is a chart showing the internal volume of the connecting tubes of two standard and two modified catheters, along with the time delays seen in the delivery of insulin infusions through these catheters.

FIG. 12 shows a preferred embodiment of the present invention on a patient's chest and situated near the right clavicle.

### DETAILED DESCRIPTION OF THE DRAWINGS

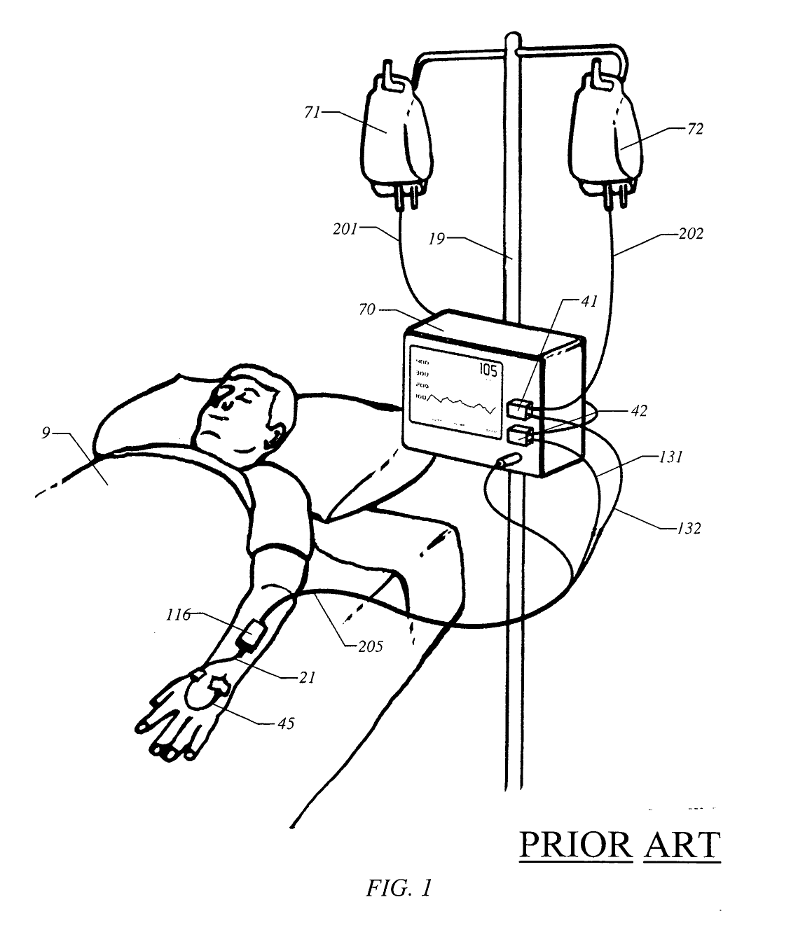

FIG. 1 illustrates the overall environment of a prior art glucose monitoring system as generally shown and described in U.S. Pat. No. 7,162,290, hereby incorporated by reference. That prior art system utilizes a similar bedside monitor with one or more similar peristaltic pumps as used with the present invention. The system of the '290 patent was strictly a blood glucose sampling and monitoring system. No insulin was infused, no multi-lumen central venous catheter was utilized, and no mention or reference to minimizing purge volume was made. A hospitalized patient 9 typically in an ICU is shown with a testing unit 116 attached to the arm by a Coban elastic band. A catheter 45 is shown inserted into a patient blood vessel on the back of the patient's hand. An infusion line 21 connects catheter 45 to testing unit 116. Fluid sources 71 and 72 for two-point calibration are suspended from a support 19. The fluid alternately passes downward through lines 201 and 202 to peristaltic pumps 41 and 42 carried in monitor housing 70. The infusion lines continue downward from the peristaltic pumps to join a three-part cable 205 which carries two lumens for fluid and one for shielded wires. The fluid lines from the peristaltic pumps are united with fluid lines of the cable by color coded Luer locks at 231 and 232. Peristaltic pumps at 41 and 42 operate in either the forward or reverse mode.

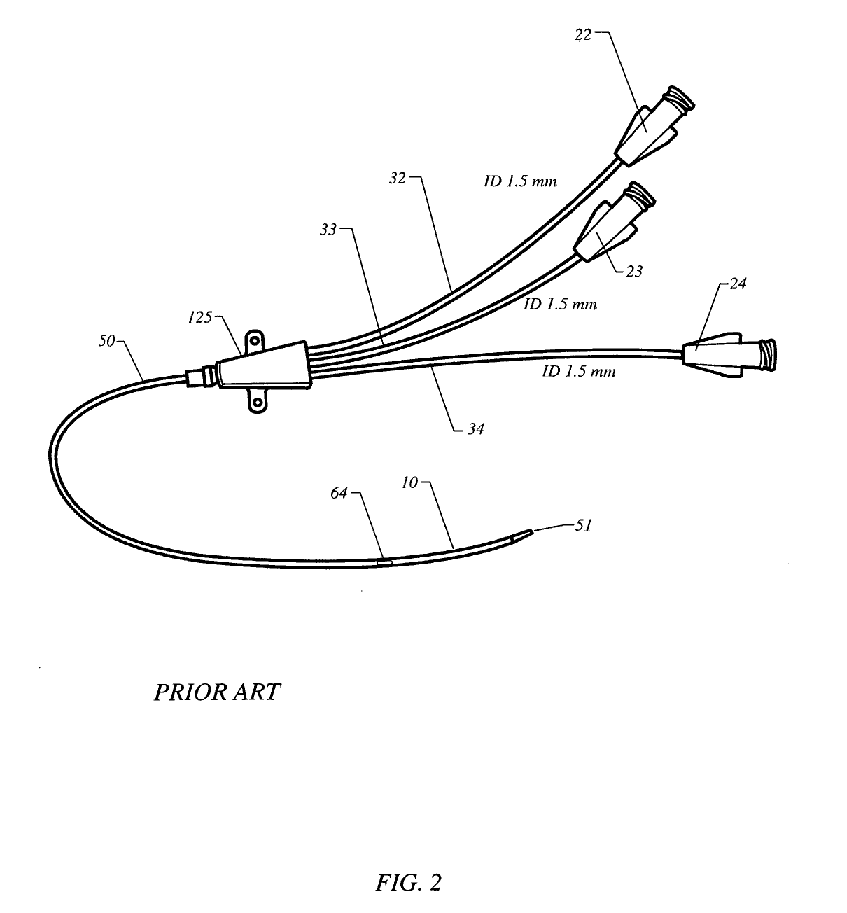

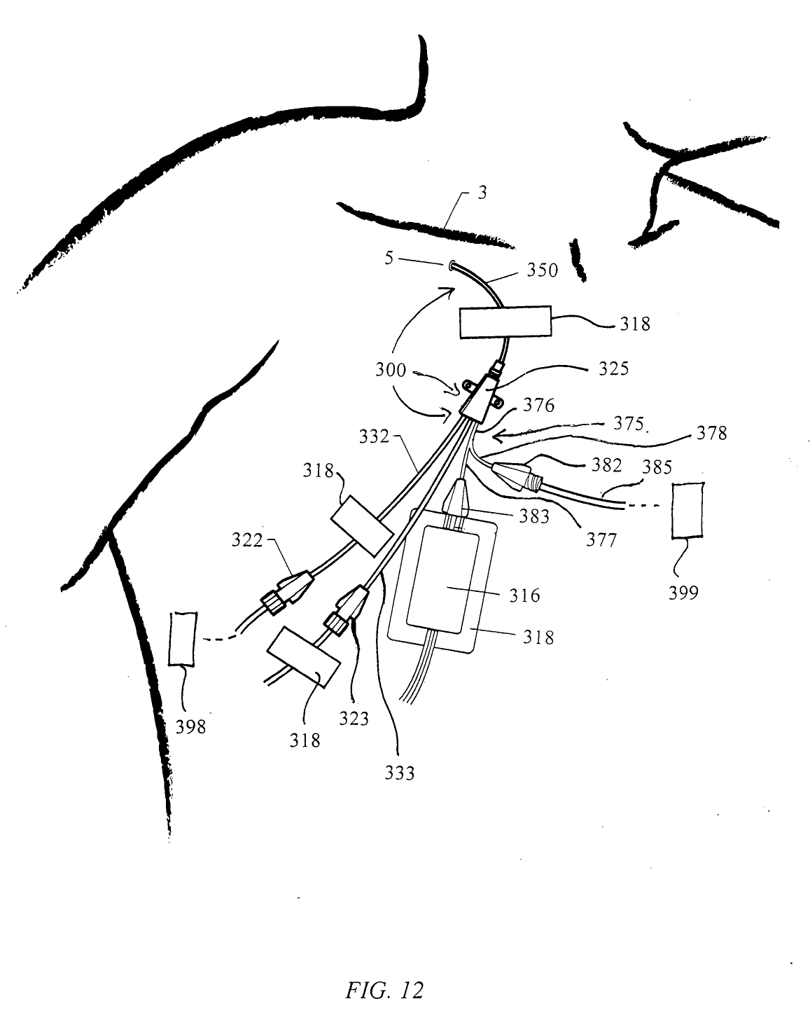

As noted above, the present invention (shown best in FIG. 12) requires modification of conventional multi-lumen catheter design in order to reduce purge volume and to facilitate the use of one single lumen for insulin infusion and blood sampling. FIGS. 2-11 and the following description thereof describes the development of the modifications made to achieve the present invention.

FIG. 2 shows the standard prior-art multi-lumen central venous catheter which is commonly used in hospital intensive care units. The inserted portion 50 carries a central lumen with a tip aperture 51 and two side lumens with apertures about 2 and 4 centimeters from the tip of the catheter. The proximal side aperture is shown as 64. Clinical blood sampling can be done with accurate results only when the proximal aperture 64 and its lumen are used for obtaining blood samples. This is because a fluid infusion upstream of the sampling aperture could contaminate the sample and cause a dilutional inaccuracy. The normal internal diameter of all three connecting tubings 32, 33 and 34 is from 1.5 to 1.75 mm. The normal length of the connecting tubings from the body of the catheter 125 to their termination at a female Luer fitting is 13 to 15 cm. Normal fluid volume of tubing 34 in an Edwards Life Sciences catheter is 0.4 mL, and for an Arrow International catheter, the volume is 0.3 mL.

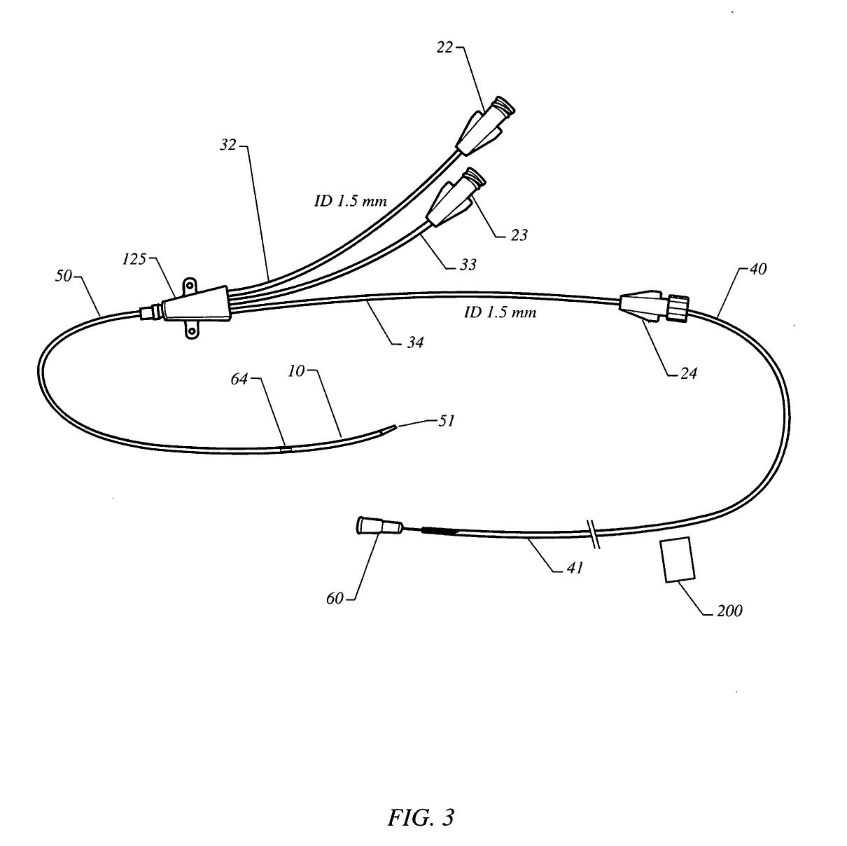

FIG. 3 shows the test set-up for determining the purge volume of standard and modified catheters. Because of intermixing of blood and clear fluid at their interface, the volume of purge fluid will always be greater than the volume of the blood sample. A standard catheter (Arrow International or Edwards Life Sciences) is shown in FIG. 3. During a test the tip of the catheter is dipped into a small vial of blood (not shown) with care taken that the proximal aperture 64 is below the liquid surface during the withdrawal of a blood sample. A 5-mL syringe (not shown) is filled with clear fluid and attached to a #20 needle 60 which is inserted into a length of Tygon tubing 40 with an ID of 0.75 mm. The syringe plunger is now pushed until a few drops of clear fluid emerge from the proximal aperture 64. The distal portion of the catheter 10 is now inserted into the vial, and blood is withdrawn past an LED/photo detector pair 200 which would be located inside an actual glucose sensor. Maximum opacity in the sampling area indicates that a pure blood sample is now in the test area. Segment 41 of the line is actually longer than shown to allow intermixed blood and fluid in the line to be pulled well beyond the test area. The plunger of the syringe is now pushed to empty the line of blood and fluid. The line is purged when perfectly clear fluid again exits from the proximal aperture of the catheter. Due to blood/fluid intermixing, the amount of fluid needed to clear the line is always greater than the volume of blood which is withdrawn. When using a standard multi-lumen catheter as shown in FIG. 2 or 3, the removal of 0.4 mL of blood requires about 3 mL of extra fluid to clear the line.

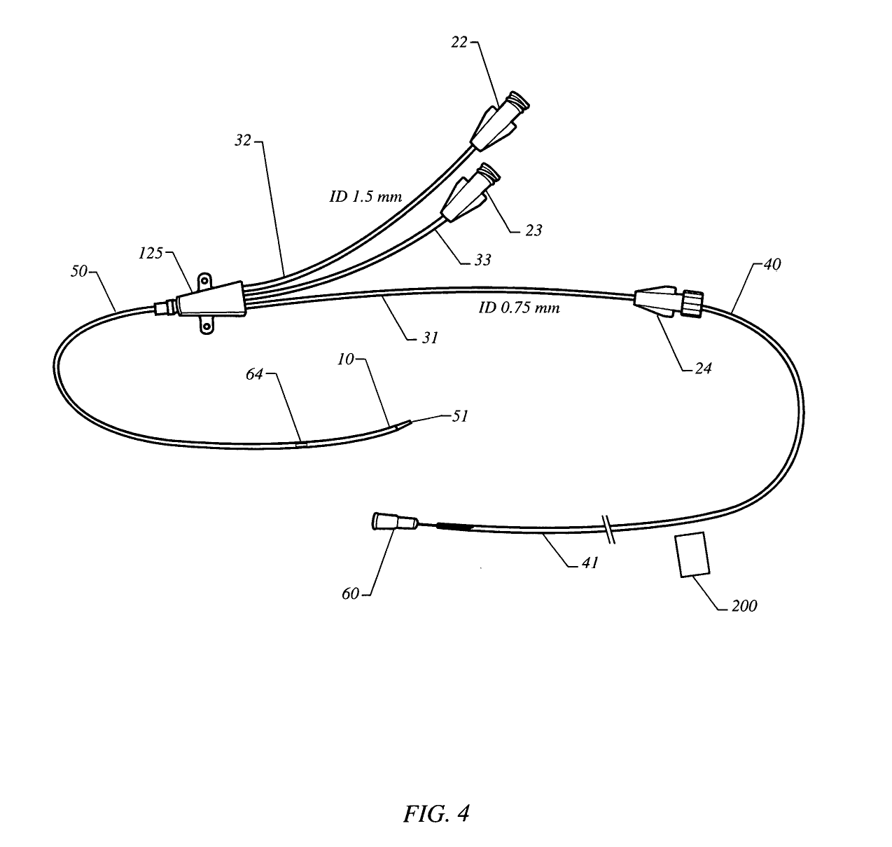

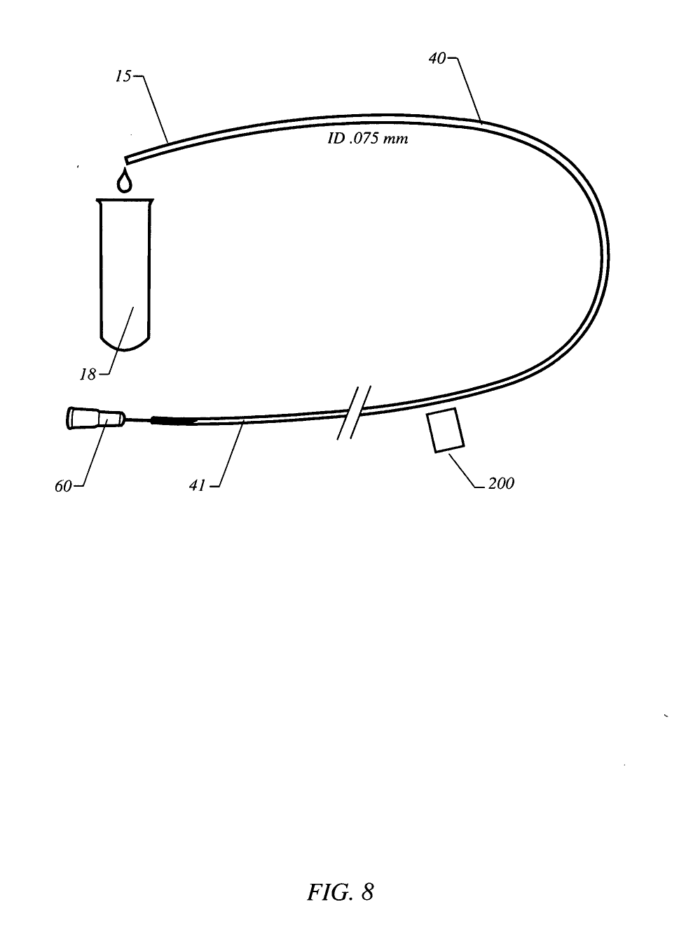

FIG. 4 shows the test set-up of FIG. 3 as used to test a first catheter modification in which the internal diameter of the connecting tube 31 is reduced from 1.5 mm to 0.75 mm. The standard length of the connecting tube or “tail” is retained at 13 cm. The purge volume is reduced by about half, as compared to the standard triple lumen catheter. (Chart—FIG. 8)

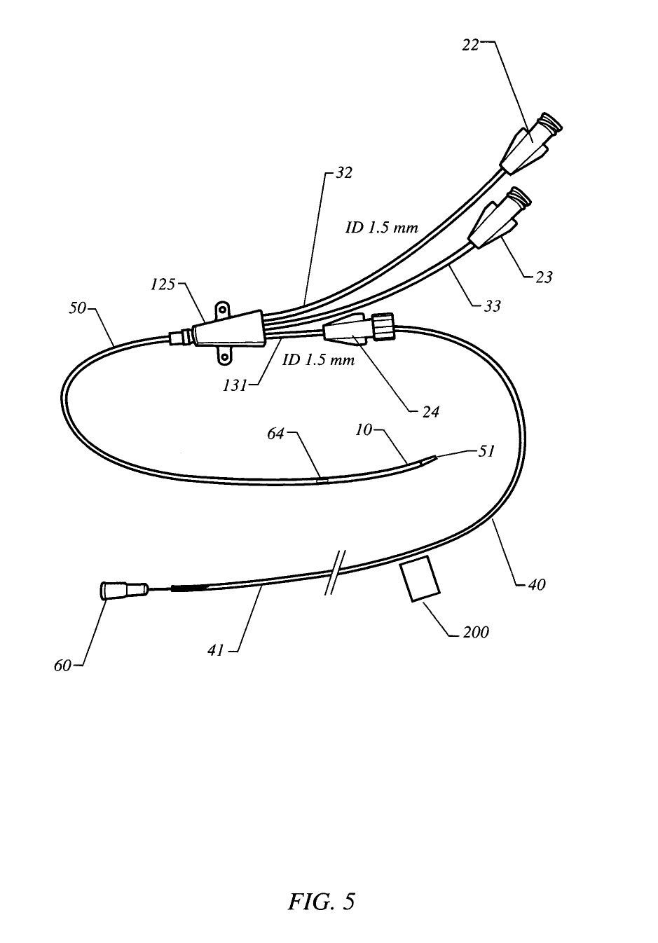

FIG. 5 shows the same test set-up as in FIG. 3 except that a second catheter modification is drawn in which the connecting tube 131 to the proximal aperture is shortened from 13 cm to 3 cm. The normal ID of 1.5 mm is retained. This modification again reduces the purge volume by about half. (Chart—FIG. 8)

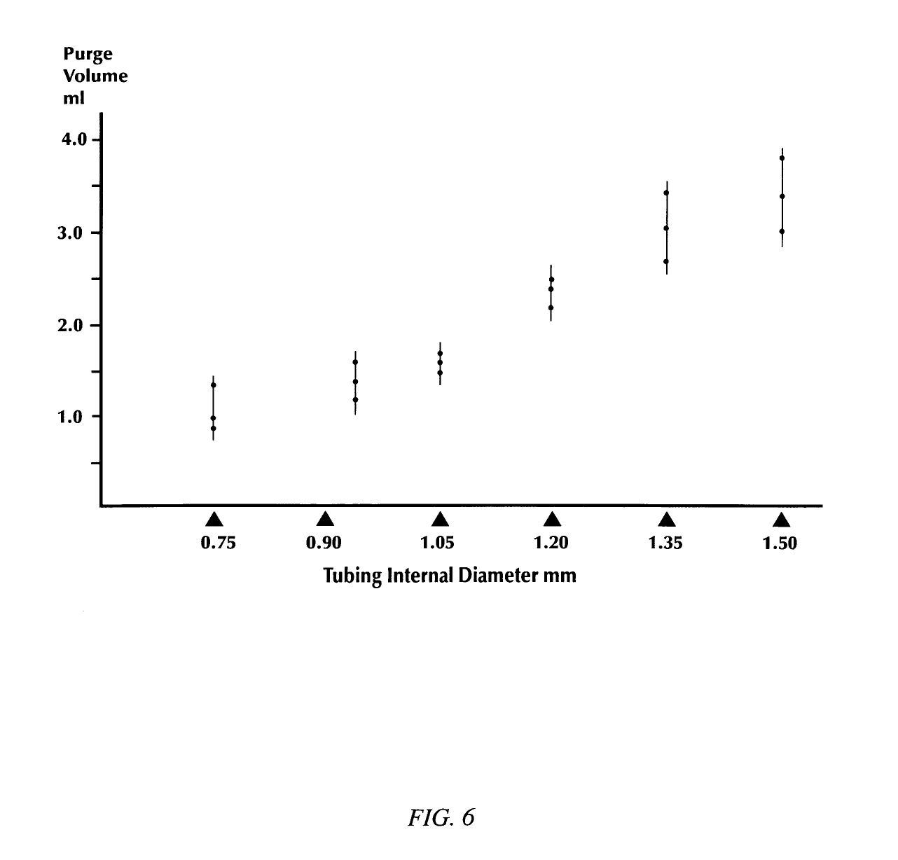

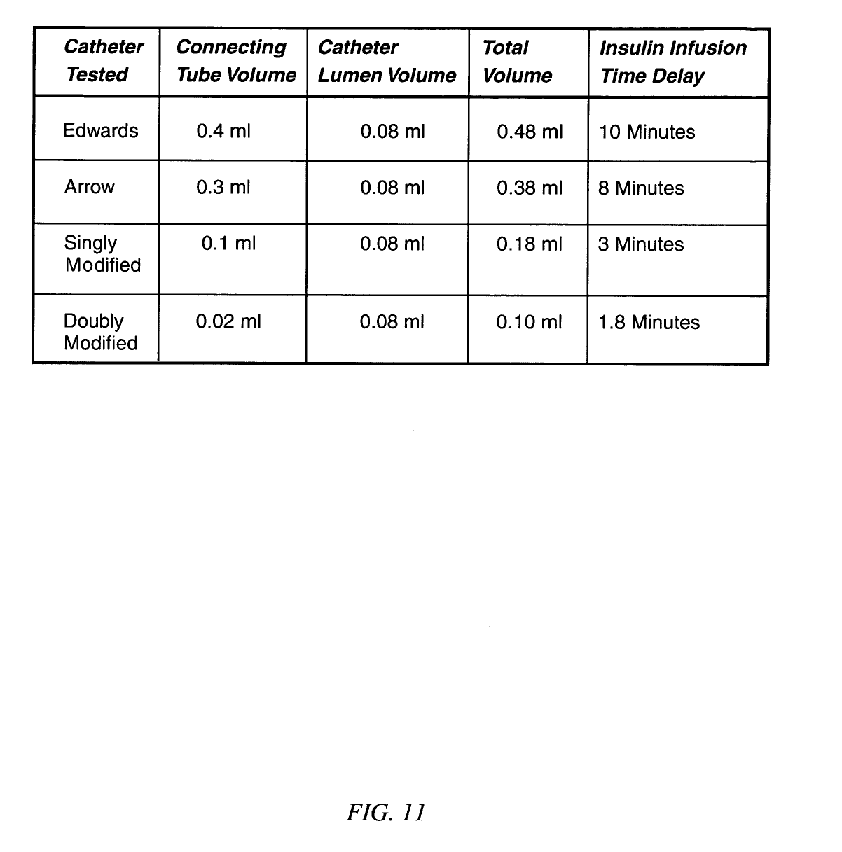

FIG. 6 shows the test results from increasing in steps the ID of a normal catheter tubing from 0.75 mm to 1.50 mm. The length of the test tubing was the standard 13 cm. There was little difference up to 1.05 mm after which the purge volume rose rapidly with the maximum value at 1.50 mm. The results are consistent with the increase in tubing volume due to the rising cross sectional area of the tubing. A 13-cm length of tubing with the minimum ID of 0.75 mm was found to hold 0.115 mL of fluid, in contrast to a standard connecting tube which holds between 0.3 and 0.4 mL. (FIG. 11)

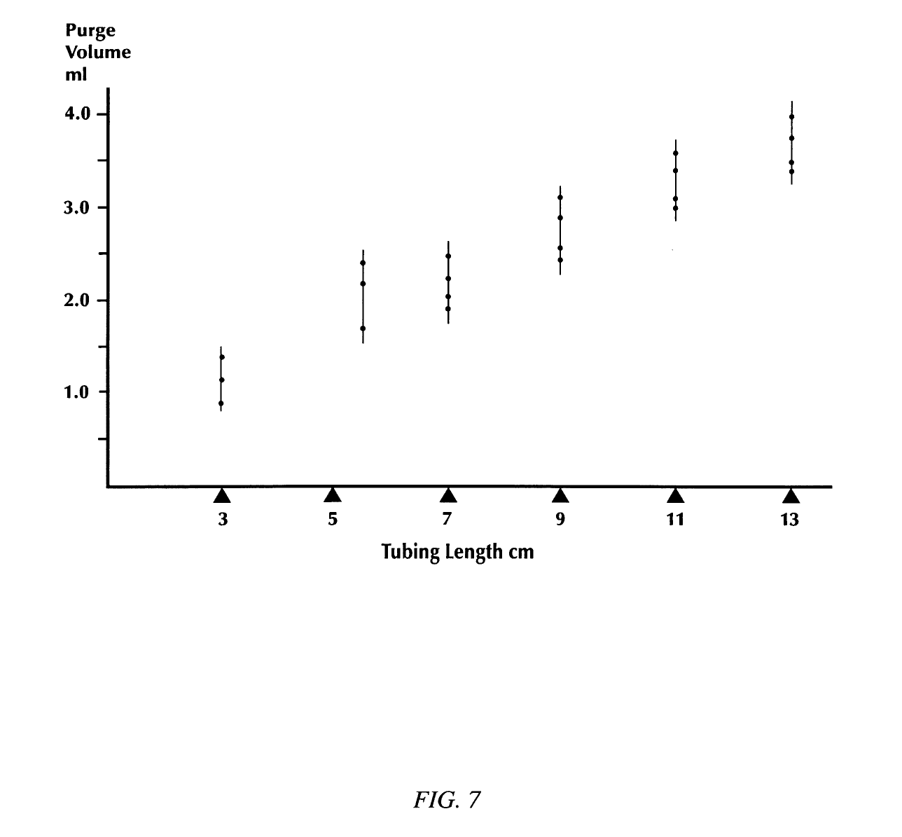

FIG. 7 shows the effect on purge volume from increasing the length of a 1.5 mm ID connecting tube in steps from 3.0 cm to 13 cm. The effect was gradual and linear with maximum purge volume at the standard length of 13 cm. A 3-cm length of connecting tubing with an ID of 1.5 mm was found to hold 0.1 mL of fluid, in contrast to a standard connecting tube which holds between 0.3 and 0.4 mL. (FIG. 11)

The data of FIGS. 6 and 7 show that purge volume is a function of the internal fluid volume of the connecting tube of the catheter lumen through which blood samples are expelled. The amount of purge fluid required can be greatly reduced by either shortening the tubing or narrowing its internal diameter, or both. Most of the excess purge fluid can be eliminated by reducing the connecting tube volume to approximately 0.1 mL.

FIG. 8 shows the set-up used to determine the theoretical minimum purge volume. A 4-foot length of Tygon tubing 40 has an optical sensor 200 placed about 20 cm from its distal end 15. The syringe is filled with fluid to 3 mL. Blood from the vial 18 is drawn into the tubing about 20 cm above the optical sensor assuring that a pure sample is present for analysis. All factors which could increase the purge volume such as Luer connectors or changes in tubing diameter or shape have been removed. The purge volume required to clear all blood from the line, due to unavoidable mixing of blood and fluid, is still from 0.5 to 0.8 mL, which is considered to be the theoretical minimum.

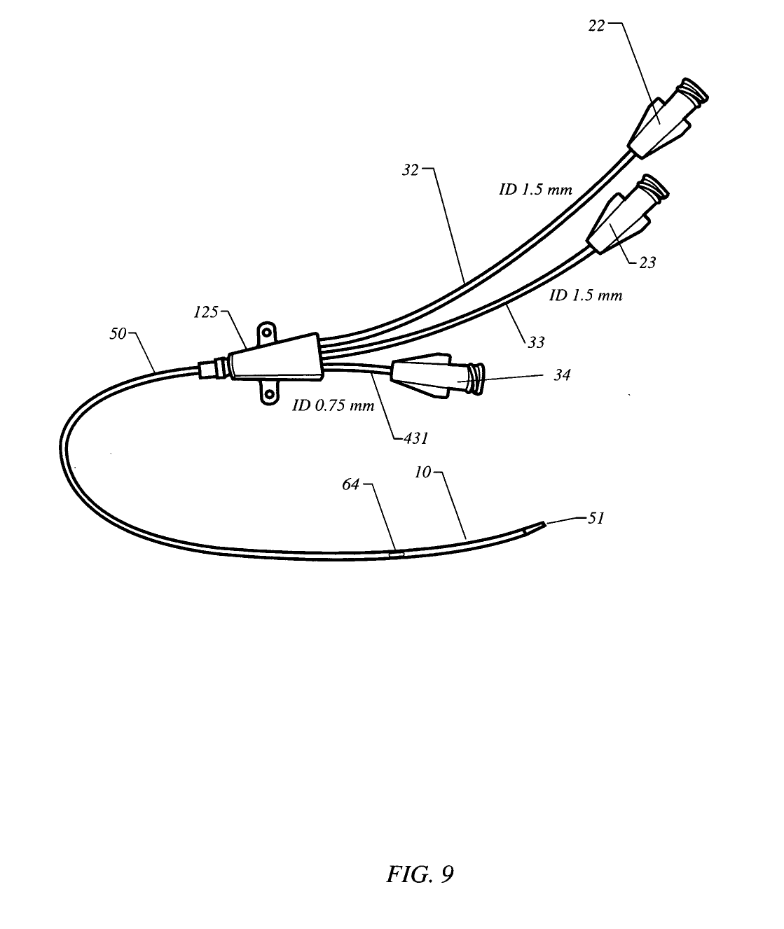

FIG. 9 shows the doubly modified catheter after both described modifications to the “tail” 431 of the catheter have been instituted. The ID of the connecting tube 431 is reduced to 0.75 mm and the length of the tube is reduced to 3 cm. Purge volume is 0.6 to 0.7 mL which is close to the theoretical minimum.

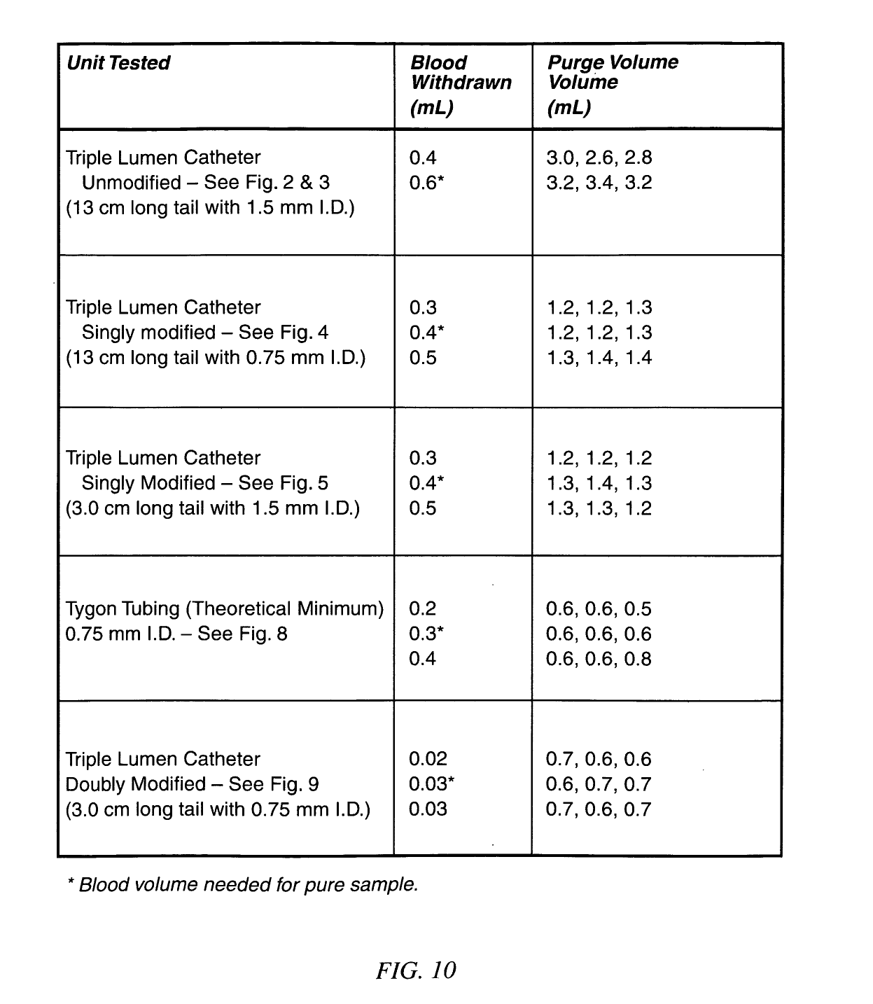

FIG. 10 is a chart which summarizes the test results from the catheter modifications to reduce purge volume. The maximum reduction is achieved by both shortening the connecting tube to 3 cm as shown in FIGS. 6 and 7 and reducing its ID to the same as the catheter lumen, which is 0.75 mm. Connecting tube volume of the doubly modified catheter is 0.02 mL or less.

FIG. 11 shows the fluid volume of the connecting tubes of two standard catheters, and of singly and doubly modified catheters as shown in FIGS. 4 and 9. Fluid that is in the connecting tube after sampling plus that fluid left in the catheter body and lumen must exit the catheter before insulin can again enter the patient's circulation. The actual time delays when infusing insulin at a rate of 3 mL/hour were tested using colored fluid. The catheters were first filled with clear fluid. Colored fluid was then injected through the female Luer fittings at the proximal end of each catheter “tail.” As expected, there was a greater delay in the appearance of colored fluid at the proximal catheter aperture of the Edwards or Arrow catheters than when testing with a catheter with the described modifications. Colored fluid appeared after 1.8 minutes with the doubly modified catheter versus 8 to 10 minutes for the two standard catheters.

FIG. 12 shows the preferred embodiment of the invention as used on a patient's upper chest for the dual purpose of withdrawing blood samples for glucose testing and for the infusion of insulin. An intravascular, multi-lumen catheter is shown generally as 300 and having a body 325. The inserted (or first) part 350 of the catheter 300 enters the subcutaneous space under the right clavicle 3 through a trocar hole 5. The opening quickly closes around the catheter and is generally covered with a small dressing. The tip of the catheter has been directed into the subclavian vein over a guide wire, which is removed after insertion of the catheter. The inserted portion of catheter 350 is identical to the inserted part 50 shown in FIG. 2, as has a proximal lumen identical to lumen 64 of FIG. 2. Fluid and electric lines 205 connect the glucose sensor 116 to a nearby bedside monitor. The unit is held to the patient by tapes 318.

A first Y-shaped connecting tubing 375 has a stem end 376, a first arm 377, a second arm 378, with the stem end 376 in fluid communication with, and extending outwardly from, the body 325 of catheter 300.

The first arm 377 of first connecting tubing 375 is connected to testing unit 316 through a luer fitting 383, forming a fluid connection between the stem end 376 and testing unit 316.

The second arm 378 of first connecting tubing 375 is connected through tubing 385 to a source 399 of insulin infusion fluid, forming a fluid connection between stem end 376 and the source 399 (shown schematically) of insulin infusion fluid.

The multi-lumen catheter 300 has a second connecting tubing 332 extending outwardly from body 325 to form a fluid connection with a source 398 (shown schematically) of one other fluid (such as blood, antibiotics, etc.) to be infused into the patient through a different lumen of catheter 300. A third connecting tubing 333 extends outwardly from body 325 and is connected to a third source (not shown) of infusion fluid.

As shown by FIGS. 2-11 and the above description thereof, the combined internal volume of stem 376 and first arm 377 is 0.1 mL or less and preferably 0.02 mL or less to minimize the cycle time. The cycle time for withdrawing a blood sample, testing and purging is completed in 3.0 minutes for a combined internal volume of 0.1 mL and 1.8 minutes for a combined internal volume of 0.02 mL.

The patient shown in FIG. 12 is illustrated without the bedside monitor and reversible peristaltic pump as shown in FIG. 1 for clarity. Although the subclavian vein is preferred, other central veins may alternately be utilized.

The invention also includes the method of providing a first passageway (i.e. stem 376 and first arm 377) extending from catheter body 325 to testing unit 316, wherein the passageway has an internal volume of 0.1 mL or less and preferably 0.02 mL or less.

The method also includes providing a second fluid passageway (i.e. stem 376 and arm 378) that intersects with and in fluid communication with the first fluid passageway by being in fluid communication with stem 376. The second fluid passageway is connected (by tubing 385) to a continuous source 399 of insulin solution. The second fluid passageway also has a combined internal volume (i.e. of stem 376 and second arm 378) of 0.1 mL or less and preferably 0.02 mL or less.

The method includes the further step of periodically actuating the reversible peristaltic pump (41 or 42 of FIG. 1) to withdraw a blood sample into testing unit 316, testing the sample, and pumping said sample and a known volume of purge fluid through the first passageway (arm 377 and stem 376) back into the patient.

The method also includes the step of continuously infusing insulin through the second passageway through the same one lumen of catheter inserted part 350 used to withdraw blood samples, wherein the combined internal volume of the second passageway (i.e. stem 376 and arm 378) is 0.1 mL or less and preferably 0.02 mL or less.

The method also includes the step of interrupting the continuous infusion of insulin for less than 3.0 minutes and preferably less than 1.8 minutes to withdraw, test and return the blood sample to the patient.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

What is claimed is:

1. In an apparatus for automatically and periodically sampling and testing blood glucose from blood samples withdrawn through an intravascular multi-lumen catheter inserted into the subclavian or other central vein of a patient, wherein a small testing unit is in fluid communication with said catheter, wherein said testing unit is cleared of blood after each test with a known purge volume of purging fluid, wherein a bedside monitor has at least one reversible peristaltic pump for either pumping blood samples outwardly from said patient into said testing unit, or pumping said blood samples from said testing unit inwardly and back into said patient through said catheter, the improvement comprising:
said intravascular multi-lumen catheter having a body and having a first part that is insertable into the subclavian or other central vein of said patient,
said multi-lumen catheter having a first Y-shaped connecting tubing having a stem end, a first arm and a second arm, with said stem end being in fluid communication with, and extending outwardly from said body,
said first arm being connected to said testing unit and forming a fluid connection between said stem end and said testing unit,
said second arm being connected to a source of insulin infusion fluid and forming a fluid connection between said stem end and said source of insulin infusion fluid,
said multi-lumen catheter having at least a second connecting tubing that extends outwardly from said body to form a fluid connection with a source of one other fluid to be infused into said patient through said intravascular catheter,
said stem and said first arm of said first Y-shaped connecting tubing having a combined internal volume of 0.1 mL or less,
wherein insulin is continuously infused through said stem of said first connecting tubing into said patient, except when blood samples are being withdrawn from said patient through said stem and said first arm of said first connecting tubing and then tested.

2. In the apparatus of claim 1, wherein each glucose testing cycle, including withdrawal of a blood sample, testing and purging are all completed in 3.0 minutes or less.

3. In the apparatus of claim 1, wherein both said stem and said first arm and said stem and said second arm of said first connecting tubing each have a combined internal volume of 0.02 mL or less.

4. In the apparatus of claim 3, wherein each glucose testing cycle, including withdrawal of a blood sample, testing and purging are all completed in less than 1.8 minutes.

5. A method for continuously infusing insulin solution through, and for periodically withdrawing and testing blood samples from, one lumen of a multi-lumen intravascular catheter connected to the subclavian or other central vein of a patient, wherein a small testing unit is worn by the patient near the body of said catheter, wherein the testing unit is cleared of blood after each test with a known volume of purge fluid, wherein a bedside monitor has at least one reversible peristaltic pump for either pumping blood samples outwardly from said catheter into said testing unit or pumping said blood samples from said testing unit inwardly and back into said patient through said catheter, and wherein a source of insulin solution is provided, comprising the steps:
providing a first fluid passageway extending from said catheter body to said testing unit, wherein said first fluid passageway has a volume of 0.1 mL or less,
providing a second fluid passageway that intersects with and in fluid communication with said first fluid passageway, and wherein said second fluid passageway is connected to said continuous source of insulin solution,
periodically actuating said reversible peristaltic pump to withdraw a blood sample through said one lumen of said multi-lumen catheter and through said first passageway into said testing unit,
testing said withdrawn blood sample in said testing unit,
pumping said blood sample from testing unit along with a known volume of purge fluid through said first passageway and said one lumen back into said patient,
continuously infusing insulin solution through said second passageway and through said one lumen of said multi-lumen catheter into said patient wherein said second passageway has an internal volume of 0.1 mL or less,
interrupting said continuous infusion of insulin for 3.0 minutes or less to withdraw, test and return said blood sample to said patient.

6. The method of claim 5 wherein each of said first and second fluid passageways extending from said catheter body has an internal fluid volume of 0.02 mL or less.

7. The method of claim 6, wherein said interruption of said infusion of less is less than 1.8 minutes to withdraw, test and return said blood sample to said patient.
